(19) United States
(12) Patent Application Publication
(10) Pub. No.: US 2010/0190240 A1
(43) Pub. Date: Jul. 29, 2010

(54) REAGENTS FOR NUCLEIC ACID PURIFICATION

(75) Inventors: Yun Jiang, Carlsbad, CA (US); Lendell L. Cummins, San Diego, CA (US); Steven A. Hofstadler, Vista, CA (US)

Correspondence Address: Casimir Jones, S.C., 2275 Deming Way, Suite 310, Madison, WI, 53562, US

(73) Assignee: IBIS BIOSCIENCES, INC.

(21) Appl. No.: 12/532,344

(22) PCT Filed: Mar. 21, 2008
(86) PCT No.: PCT/US08/57901

§ 371 Date: Mar. 15, 2010

Related U.S. Application Data
(60) Provisional application No. 60/919,212, filed on Mar. 21, 2007.

(51) Int. Cl. C12N 1/08; C07H 21/00; C07H 21/02; C07H 21/04

(52) U.S. Cl. 435/270; 536/25.41; 536/25.42; 536/23.1

(57) ABSTRACT

Embodiments of the present invention provide methods and kits for purifying nucleic acids. In particular, embodiments of the present invention provide methods and kits for purifying nucleic acids through the use of magnetic particles in binding buffers.

This application is a U.S. National Phase application under 35 U.S.C. §371 claiming priority to International Application Number PCT/US2008/057901 filed on Mar. 21, 2008 under the Patent Cooperation Treaty, which claims the benefit of priority to U.S. Provisional Application Ser. No. 60/919,212, filed Mar. 21, 2007, the disclosure of which is incorporated by reference in its entirety for any purpose.

### FIELD OF INVENTION

Embodiments of the present invention provide methods and kits for purifying nucleic acids. In particular, embodiments of the present invention provide methods and kits for purifying nucleic acids through the use of magnetic particles in binding buffers.

### BACKGROUND OF INVENTION

The techniques of molecular biology often require the purification of nucleic acids away from other compounds including lipids, polysaccharides and proteins. Selection of a given method of purification depends on the desired quantity of the target nucleic acid, its molecular weight, the purity needed for subsequent use, and the available time and expense per sample. While many approaches have been devised for nucleic acid purification from diverse starting materials, for example, plant and animal tissue or prokaryotic samples, most suffer one or more shortcomings including low yield, contamination from reagents used for purification, reagent toxicity to operators, inefficiency, or degradation of the target nucleic acid.

Use of coated magnetic beads to bind nucleic acids in a reaction mixture offers several advantages including, for example, avoidance of centrifugation or vacuum processing, operator safety, and high purity. Using this technique, samples are lysed and incubated with a binding buffer. After addition of the magnetic beads, nucleic acids released from the samples are bound to the bead surface. Unbound contaminants are removed in subsequent washing steps. Thereafter, the purified nucleic acid is eluted from the beads with a low salt elution buffer. The purified nucleic acid may then be used in a variety of applications including, for example, PCR, restriction digestion and Southern blotting. Importantly, use of magnetic beads for nucleic acid purification is limited by the recovery yield of available protocols, and the speed and complexity of the isolation procedure. Thus, methods and kits for nucleic acid purification using magnetic beads are needed that provide a faster isolation procedure, and greater nucleic acid recovery, from a diversity of starting materials.

### SUMMARY OF INVENTION

Embodiments of the present invention provide methods and kits for purifying nucleic acids. In particular, embodiments of the present invention provide methods and kits for purifying nucleic acids through the use of magnetic particles in binding buffers.

In one aspect, for example, the invention relates to methods for nucleic acid purification. In certain embodiments, the methods include a) combining a binding buffer comprising polyoxyethylene sorbitan monolaurate, at least one alcohol and at least one salt with at least one paramagnetic particle (e.g., a carboxyl coated paramagnetic particle, a silica based paramagnetic particle, or the like) to generate a suspension; b) combining at least one sample comprising at least one nucleic acid with said suspension, wherein said paramagnetic particle reversibly captures said nucleic acid (e.g., said nucleic acid non-covalently binds to said paramagnetic particle or the like) to generate a combination comprising said paramagnetic particle with said captured nucleic acid; and c) separating said paramagnetic particle with said captured nucleic acid from one or more other components of the combination using a magnetic separator, thereby purifying said nucleic acid. In some embodiments, the methods of the invention include washing said paramagnetic particle with said captured nucleic acid with a wash buffer. In certain embodiments, the methods include combining said sample with a lysis buffer to generate a lysate. In these embodiments, generally b) comprises combining said lysate with said suspension.

Typically, the methods described herein include releasing said captured nucleic acid from said paramagnetic particle to generate released nucleic acid. In some embodiments, for example, said releasing comprises incubating said paramagnetic particle with said captured nucleic acid with an elution buffer. The methods also generally include separating said released nucleic acid from said paramagnetic particle using said magnetic separator.

To further illustrate, embodiments of the present invention provide methods for nucleic acid purification, comprising one or more steps of: a) obtaining a sample comprising or suspected of comprising at least one nucleic acid; b) providing: i) a solution comprising at least one paramagnetic particle (e.g., a carboxyl coated paramagnetic particle, a silica based paramagnetic particle, or the like); ii) a solution comprising a binding buffer comprising polyoxyethylene sorbitan monolaurate, at least one alcohol and at least one salt; iii) a lysis buffer; iv) a magnetic separator; v) a wash buffer; and vi) an elution buffer; and c) combining the binding buffer with at least one paramagnetic particle to generate a suspension; d) combining the sample with the lysis buffer to generate a lysate; e) combining the suspension with the lysate to generate a combination; f) placing the combination of the suspension with the lysate into a magnetic separator; g) separating the combination of the suspension with the lysate from the at least one paramagnetic particle; h) washing the at least one paramagnetic particle with the wash buffer; i) incubating the at least one paramagnetic particle with the elution buffer; and j) separating the at least one paramagnetic particle from the elution buffer using said magnetic separator.

In some embodiments of the present invention, the solution comprising a binding buffer comprises at least 10% polyoxyethylene sorbitan monolaurate. In other embodiments, the solution comprising a binding buffer comprises at least 20% polyoxyethylene sorbitan monolaurate by volume.

Embodiments of the present invention are not limited by the nature of the alcohol used. In some embodiments, the alcohol comprises butanol, isopropanol, and/or ethanol. In other embodiments, the binding buffer comprises at least 10% ethanol by volume. In further embodiments, the binding buffer comprises at least 20% ethanol by volume. In still further embodiments, a mixture of alcohols is used.

Embodiments of the present invention are not limited by the nature of the salt used. In some embodiments, the salt comprises lithium chloride, lithium perchlorate, potassium chloride, sodium bromide, potassium bromide, cesium chloride, ammonium acetate and/or sodium chloride. In other embodiments, the binding buffer comprises at least 1.0 M sodium chloride. In further embodiments the binding buffer comprises at least 2.0 M sodium chloride. In preferred embodiments the binding buffer comprises at least 2.0 M sodium chloride, and at least 10% polyoxyethylene sorbitan monolaurate by volume. In particularly preferred embodiments the binding buffer comprises at least 2.0 M sodium chloride, at least 10% polyoxyethylene sorbitan monolaurate by volume, and at least 10% ethanol by volume. In some embodiments, a mixture of salts is used.

In some embodiments of the present invention, the combination of the suspension with the lysate comprises at least 7.5% polyoxyethylene sorbitan monolaurate. In further embodiments, the combination of the suspension with the lysate comprises at least 10% polyoxyethylene sorbitan monolaurate. In other embodiments, the combination of the suspension with the lysate comprises at least 10% polyoxyethylene sorbitan monolaurate and 1.5 M sodium chloride.

Embodiments of the present invention are not limited by the nature of the nucleic acid that is purified. In some embodiments, the at least one nucleic acid is DNA. In other embodiments, the at least one nucleic acid is RNA. In further embodiments, the at least one nucleic acid is nucleic acid from a prokaryote. In still further embodiments, the at least one nucleic acid is nucleic acid from a eukaryote. In preferred embodiments the sample is from a biologic source. In other embodiments, the sample is from a non-biological source.

In some embodiments of the present invention, the combination is a reaction mixture generated by sequentially conducting steps a) to e).

In some embodiments, the present invention provides methods for nucleic acid purification, comprising one or more of the steps of: a) obtaining a sample comprising or suspected of comprising at least one nucleic acid; b) providing: i) a solution comprising at least one paramagnetic particle (e.g., a carboxyl coated paramagnetic particle, a silica based paramagnetic particle, or the like); ii) a solution comprising a binding buffer comprising at least one polyoxyethylene sorbitan, at least one alcohol and at least one salt; iii) a lysis buffer; iv) a magnetic separator; v) a wash buffer; and vi) an elution buffer; and c) combining the binding buffer with at least one paramagnetic particle to generate a suspension; d) combining the sample with the lysis buffer to generate a lysate; e) combining the suspension with the lysate to generate a combination; f) placing the combination of the suspension with the lysate into a magnetic separator; g) separating the combination of the suspension with the lysate from the at least one paramagnetic particle; h) washing the at least one paramagnetic particle with the wash buffer; i) incubating the at least one paramagnetic particle with the elution buffer; and j) separating the at least one paramagnetic particle from the elution buffer using said magnetic separator. Embodiments of the present invention are not limited by the nature of the polyoxyethylene sorbitan used. In some embodiments, the polyoxyethylene sorbitan comprises polyoxyethylene sorbitan monolaurate, polyoxyethylene sorbitan monopalmitate, and/or polyoxyethylene sorbitan monostearate.

In some embodiments, the present invention further provides kits comprising one or more of: a) a binding buffer, comprising: i) polyoxyethylene sorbitan monolaurate; and at least one alcohol; and b) at least one paramagnetic particle (e.g., a carboxyl coated paramagnetic particle, a silica based paramagnetic particle, or the like); c) a lysis buffer; d) a reaction vessel; e) a magnetic separator; f) a wash buffer; and d) an elution buffer. In some embodiments, the binding buffer comprises at least 10% polyoxyethylene sorbitan monolaurate by volume. In other embodiments, the binding buffer comprises at least 20% polyoxyethylene sorbitan monolaurate by volume. In further embodiments, the at least one alcohol comprises ethanol. In still further embodiments, the at least one alcohol comprises at least 10% ethanol by volume. In preferred embodiments the at least one alcohol comprises at least 20% ethanol by volume.

In some embodiments of the present invention, the binding buffer further comprises at least one salt. In further embodiments, the at least one salt is sodium chloride. In preferred embodiments, the at least one salt comprises at least 1.0 M sodium chloride. In particularly preferred embodiments, the at least one salt comprises at least 2.0 M sodium chloride. In other embodiments, the binding buffer comprises at least 2.0 M sodium chloride and at least 10% polyoxyethylene sorbitan monolaurate by volume. In still further embodiments, the binding buffer comprises at least 2.0 M sodium chloride, at least 10% polyoxyethylene sorbitan monolaurate by volume, and at least 10% ethanol by volume. In some embodiments, the wash buffer is 70% ethanol.

In some embodiments the kit further comprises instructions for using the kit on a computer readable medium. Instructions include, but are not limited to, instructions for mixing buffers with the sample, use of control samples, carrying out experiments, reading data, interpreting data, analyzing data and transmitting data. Instructions may include those items required by regulatory institutions for use of the kit as an in vitro diagnostic product or other type of product.

In some embodiments of the present invention the binding buffer, the at least one paramagnetic particle, the lysis buffer, the wash buffer and the elution buffer are provided in individual containers. It is noted that the kit need not be configured to require a one-to-one buffer sample mixture. The buffers may be provided as 5×, 10×, etc. buffers for dilution either before or during use. In other embodiments, the wash buffer comprises 70% ethanol.

In some embodiments, the present invention further provides a composition comprising at least one paramagnetic particle (e.g., a carboxyl coated paramagnetic particle, a silica based paramagnetic particle, or the like) in a binding buffer comprising 20% polyoxyethylene sorbitan monolaurate by volume, 20% ethanol by volume, and 2.5 M sodium chloride, as well as similar compositions based on parameters described herein, or their functional equivalents.

### DEFINITIONS

To facilitate an understanding of embodiments of the present invention, a number of terms and phrases are defined below:

As used herein, the term “salt” refers to stable compound composed of a cation bound to an anion. Salts are typically formed in a chemical reaction between a base or a metal and an acid yielding a salt and water (e.g., NaOH+HCl=NaCl+H₂O). The term salts refers to but is not limited to acetates, carbonates, chlorides, cyanides, nitrates, nitrites, phosphates, and sulfates.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include urine and blood products, such as plasma, serum and the like. Such examples are not however to be construed as limiting the sample types applicable to the present invention. A sample suspected of containing a human chromosome or sequences associated with a human chromosome may comprise a cell, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

As used herein, the term “instructions for using said kit” refers to instructions for using the reagents contained in the kit for the purification of a nucleic acid in a sample. In some embodiments, the instructions further comprise the statement of intended use required by the U.S. Food and Drug Administration (FDA) in labeling in vitro diagnostic products.

As used herein, the term “subject” refers to any animal (e.g., a mammal), including, but not limited to, humans, non-human primates, rodents, and the like, which is to be the recipient of a particular diagnostic test or treatment. Typically, the terms “subject” and “patient” are used interchangeably herein in reference to a human subject.

As used herein, the term “non-human animals” refers to all non-human animals including, but are not limited to, vertebrates such as rodents, non-human primates, ovines, bovines, ruminants, lagomorphs, porcines, caprines, equines, canines, felines, ayes, etc.

The term “gene” refers to a nucleic acid (e.g., DNA) sequence that comprises coding sequences necessary for the production of a polypeptide, RNA (e.g., including but not limited to, mRNA, tRNA and rRNA) or precursor. The polypeptide, RNA, or precursor can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified,” “mutant,” “polymorphism,” and “variant” refer to a gene or gene product that displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotide or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the terms “an oligonucleotide having a nucleotide sequence encoding a gene” and “polynucleotide having a nucleotide sequence encoding a gene,” means a nucleic acid sequence comprising the coding region of a gene or, in other words, the nucleic acid sequence that encodes a gene product. The coding region may be present in a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements include splicing signals, polyadenylation signals, termination signals, etc.

As used herein, the terms “complementary” or “complementarity” are used in reference to polynucleotides (i.e., a sequence of nucleotides) related by the base-pairing rules. For example, for the sequence 5′-“A-G-T-3′,” is complementary to the sequence 3′-“T-C-A-S′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous.” The term “inhibition of binding,” when used in reference to nucleic acid binding, refers to inhibition of binding caused by competition of homologous sequences for binding to a target sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “competes for binding” is used in reference to a first polypeptide with an activity which binds to the same substrate as does a second polypeptide with an activity, where the second polypeptide is a variant of the first polypeptide or a related or dissimilar polypeptide. The efficiency (e.g., kinetics or thermodynamics) of binding by the first polypeptide may be the same as or greater than or less than the efficiency substrate binding by the second polypeptide. For example, the equilibrium binding constant (K_{D}) for binding to the substrate may be different for the two polypeptides. The term “Kₘ” as used herein refers to the Michaelis-Menton constant for an enzyme and is defined as the concentration of the specific substrate at which a given enzyme yields one-half its maximum velocity in an enzyme catalyzed reaction.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42 C when a probe of about 500 nucleotides in length is employed.

“Medium stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42 C when a probe of about 500 nucleotides in length is employed.

“Low stringency conditions” comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42 C when a probe of about 500 nucleotides in length is employed. The present invention is not limited to the hybridization of probes of about 500 nucleotides in length. The present invention contemplates the use of probes between approximately 10 nucleotides up to several thousand (e.g., at least 5000) nucleotides in length.

One skilled in the relevant understands that stringency conditions may be altered for probes of other sizes (See e.g., Anderson and Young, Quantitative Filter Hybridization, in Nucleic Acid Hybridization [1985] and Sambrook et al., Molecular Cloning: A Laboratory Manual, Cold Spring Harbor Press, NY [1989]).

The following terms are used to describe the sequence relationships between two or more polynucleotides: “reference sequence”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman [Smith and Waterman, Adv. Appl. Math. 2: 482 (1981)] by the homology alignment algorithm of Needleman and Wunsch [Needleman and Wunsch, J. Mol. Biol. 48:443 (1970)], by the search for similarity method of Pearson and Lipman [Pearson and Lipman, Proc. Natl. Acad. Sci. (U.S.A.) 85:2444 (1988)], by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence.

The term “polymorphic locus” is a locus present in a population that shows variation between members of the population (i.e., the most common allele has a frequency of less than 0.95). In contrast, a “monomorphic locus” is a genetic locus at little or no variations seen between members of the population (generally taken to be a locus at which the most common allele exceeds a frequency of 0.95 in the gene pool of the population).

As used herein, the term “genetic variation information” or “genetic variant information” refers to the presence or absence of one or more variant nucleic acid sequences (e.g., polymorphism or mutations) in a given allele of a particular gene.

As used herein, the term “detection assay” refers to an assay for detecting the presence of absence of specific nucleic acid sequences (e.g., polymorphisms or mutations), for example, in a given allele of a particular gene.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Qβ replicase, MDV-1 RNA is the specific template for the replicase (D. L. Kacian et al., Proc. Natl. Acad. Sci. USA 69:3038 [1972]). Other nucleic acids will not be replicated by this amplification enzyme. Similarly, in the case of T7 RNA polymerase, this amplification enzyme has a stringent specificity for its own promoters (Chamberlin et al., Nature 228:227 [1970]). In the case of T4 DNA ligase, the enzyme will not ligate the two oligonucleotides or polynucleotides, where there is a mismatch between the oligonucleotide or polynucleotide substrate and the template at the ligation junction (D. Y. Wu and R. B. Wallace, Genomics 4:560 [1989]). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press [1989]).

As used herein, the term “amplifiable nucleic acid” is used in reference to nucleic acids that may be amplified by any amplification method. It is contemplated that “amplifiable nucleic acid” will usually comprise “sample template.”

As used herein, the term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target,” refers to a nucleic acid sequence or structure to be detected or characterized. Thus, the “target” is sought to be sorted out from other nucleic acid sequences. A “segment” is defined as a region of nucleic acid within the target sequence.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

As used herein the term “coding region” when used in reference to structural gene refers to the nucleotide sequences that encode the amino acids found in the nascent polypeptide as a result of translation of a mRNA molecule. The coding region is bounded, in eukaryotes, on the 5′ side by the nucleotide triplet “ATG” that encodes the initiator methionine and on the 3′ side by one of the three triplets, which specify stop codons (i.e., TAA, TAG, TGA).

As used herein, the term “purified” or “to purify” refers to the removal of one or more contaminants or components from a sample.

The term “recombinant DNA molecule” as used herein refers to a DNA molecule that is comprised of segments of DNA joined together by means of molecular biological techniques.

The term “recombinant protein” or “recombinant polypeptide” as used herein refers to a protein molecule that is expressed from a recombinant DNA molecule.

The term “native protein” as used herein to indicate that a protein does not contain amino acid residues encoded by vector sequences; that is the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four consecutive amino acid residues to the entire amino acid sequence minus one amino acid.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 [1989]).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al., supra, pp 7.39-7.52 [1989]).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

The term “transgene” as used herein refers to a foreign, heterologous, or autologous gene that is placed into an organism by introducing the gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene. The term “autologous gene” is intended to encompass variants (e.g., polymorphisms or mutants) of the naturally occurring gene. The term transgene thus encompasses the replacement of the naturally occurring gene with a variant form of the gene.

As used herein, the term “vector” is used in reference to nucleic acid molecules that transfer DNA segment(s) from one cell to another. The term “vehicle” is sometimes used interchangeably with “vector.”

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term “host cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

The terms “overexpression” and “overexpressing” and grammatical equivalents, are used in reference to levels of mRNA to indicate a level of expression approximately 3-fold higher than that typically observed in a given tissue in a control or non-transgenic animal. Levels of mRNA are measured using any of a number of techniques known to those skilled in the art including, but not limited to Northern blot analysis (See, Example 10, for a protocol for performing Northern blot analysis). Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the RAD50 mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “test compound” refers to any chemical entity, pharmaceutical, drug, and the like that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention's embodiments. A “known therapeutic compound” refers to a therapeutic compound that has been shown (e.g., through animal trials or prior experience with administration to humans) to be effective in such treatment or prevention.

### DESCRIPTION OF INVENTION

Embodiments of the present invention provide methods and kits for purifying nucleic acids. In particular, embodiments of the present invention provide methods and kits for purifying nucleic acids through the use of magnetic particles in binding buffers.

I. Methods of Purification of Nucleic Acid using Magnetic Particles

The isolation of DNA or RNA from different samples is often important for molecular testing for a variety of purposes including, for example, PCR, restriction digestion, Southern blotting and Northern blotting. Use of magnetic particles simplifies the nucleic acid isolation, thereby enabling high-throughput automation of purification. Surprisingly, in experiments conducted in the course of development of embodiments of the present invention, it was found that compared to other additives to the binding buffer (for example, polyethylene glycol or PEG), the addition of polyoxyethylene sorbitan monolaureate (TWEEN 20) resulted in greater nucleic recovery, and a faster purification procedure. While understanding the mechanism underlying the present invention is not required for the successful practice of the invention, and while in no way limiting the invention to any particular mechanism, it is believed that the use of polyoxyethylene sorbitan monolaureate in the binding buffer reduces the viscosity of the buffer. The reduced buffer viscosity increases the mobility of the magnetic particles, and results in a faster nucleic acid isolation procedure with improved yield of nucleic acid. Moreover, the embodiments of the present invention are useful for the isolation of both DNA and RNA using a single protocol. For example, in some embodiments the method of the present invention may be used for the isolation of DNA only with the addition of RNase, the isolation of RNA only with the addition of DNase, or for the isolation of both DNA and RNA.

### II. Optimization of Polyoxyethylene Sorbitan, Ethanol and NaCl in Binding Buffer

Experiments demonstrate that the addition of at least one alcohol and at least one salt to a binding buffer further comprising a polyoxyethylene sorbitan further improves the efficiency and yield of the purification. In some embodiments, the binding buffer comprises 5%-40% of an alcohol, preferably 10%-20% of ethanol, for example, 10% and 20% ethanol, although higher and lower amounts are contemplated. In other embodiments, the binding buffer comprises 0.5M-3.0 M NaCl, preferably 1.M-2.5 M NaCl, for example 1.M, 2.0M and 2.5 M NaCl, although higher and lower amounts are contemplated. In further embodiments, the binding buffer comprises 5%±40% polyoxyethylene sorbitan, preferably 10%±30% polyoxyethylene sorbitan monolaurate, for example, 20%, 25% and 30% polyoxyethylene sorbitan monolaurate, although higher and lower amounts are contemplated. In other embodiments, the vol % of polyoxyethylene sorbitan and alcohol in combination in the binding buffer is constant, for example, at 40% in combination, wherein the respective vol % of polyoxyethylene sorbitan and alcohol may vary to yield 40% in sum. In other embodiments, the combined vol % of polyoxyethylene sorbitan and alcohol is 45%, although higher and lower amounts are contemplated.

### III. Kits

As used herein, in some embodiments the term “kit” refers to any delivery system for delivering materials. In the context of nucleic acid purification, such delivery systems include systems that allow for the storage, transport, or delivery purification reagents (e.g., paramagnetic particles, positive and negative nucleic acid standards and controls, etc. in the appropriate containers, and/or other materials (e.g., buffers, written instructions for performing the assay etc.) from one location to another. For example, kits include one or more enclosures (e.g., boxes) containing the relevant reaction reagents and/or other materials. As used herein, the term “fragmented kit” refers to delivery systems comprising two or more separate containers that each contain a sub-portion of the total kit components. The containers may be delivered to the intended recipient together or separately. For example, a first container may contain a lysis buffer for use in an assay, while a second container may contain a wash buffer or an elution buffer. Indeed, any delivery system comprising two or more separate containers that each contains a sub-portion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

In some embodiments, the kits are configured to allow reactions to occur where the only thing that is added to a reaction container is a sample comprising or suspected of comprising a nucleic acid. In preferred embodiments, all the various components for running any of the sample preparation methods are included in a kit. It is appreciated that the instrumentation described herein (e.g., magnetic separator, containers, instructions on a computer readable medium) can also be sold as kit which would include the instrumentation described herein as well as a plurality of pre-ordered or ordered reagents and solutions.

In some embodiments, the kit comprises instructions, directing a user of the kit to use the kit with samples comprising or suspected of comprising at least one nucleic acid for nucleic acid purification. In some embodiments, the instructions for using the kit are provided on a computer readable medium. In further embodiments, a computer program comprising instructions directs a processor to analyze data derived from use of said buffers, reagents and instrumentation. In some embodiments, the instructions are physical components of the kits of the present invention that dictate the manipulations of physical objects and activities that, as components of the claimed kits, implement a set of actions to accomplish purification of a nucleic acid. In further embodiments, a computer-based analysis program is used to translate raw data generated by the nucleic acid purification kit into data of use to a user e.g., a concentration range, or dilution protocol.

As used herein a “computer program” is a set of statements or instructions to be used directly or indirectly in a computer in order to bring about a certain result i.e., a sequence of instructions enabling a computer to solve a problem. As used herein, a “processor” is a computer program (e.g., a compiler) that puts another program into a form acceptable to the computer. The instructions of the embodiments of the present invention are functionally related to the substrate kit. Instructions and reagents of embodiments of the present invention are interrelated, so as to produce a product useful for the purpose of nucleic acid purification. In some embodiments, the instructions of the present invention do not achieve their purpose of nucleic acid purification without the reagents (e.g., buffers, paramagnetic particles) of the kit, and the reagents of the kit do not produce the desired result without instructions.

### EXPERIMENTAL EXAMPLES

The following examples serve to illustrate certain preferred embodiments and aspects of the present invention and are not to be construed as limiting the scope thereof.

### Example 1

### Nucleic Acid Yield after Purification with 20% TWEEN 20 (Polyoxyethylene Sorbitan Monolaurate)

### Sample Origin

Experiments were performed on aliquots of human white blood cell lysate prepared from whole blood, and stored as frozen stock samples. In Example 1 the identical lysate sample was used for all comparisons

### Purification Protocol

In experimental Example 1, DNA yield using exemplary binding buffer compositions were compared using 20% TWEEN 20, and varying amounts of ethanol and salt (Table 1). The magnetic bead suspension solution was 40 microliter beads, 10 mM TRIS, and 3600 μl buffer (TWEEN buffer) for a 1:10 dilution of the beads in final buffer. The reaction mixture was 50 μL sample lysate, and 100 μL magnetic bead suspension. The mixture was incubated for 10 minutes, whereupon the beads were separated and washed 3 times with 500 μL 70% ethanol. The beads were then dried for 5 minutes before elution into 50 μL distilled water at 55° C. for 5 minutes.

TABLE 1

| | Buffer ID Number | | | | | |
|---|---|---|---|---|---|---|
| | 1 | 2 | 3 | 4 | 5 | 6 |
| Tween 20 | 20% | 20% | 20% | 20% | 20% | 20% |
| Ethanol | 0 | 0 | 10% | 10% | 20% | 20% |
| NaCl | 2.5M | 1.0M | 2.0M | 1.0M | 1.5M | 1.0M |
| DNA yield (ng/uL) | 3.7 | 1.5 | 1.5 | 15.8 | 34.4 | 29.8 |

### DNA Detection

Eluted DNA was then quantitated using a UV spectrophotometer. Absorbance at A260 wavelength was recorded. DNA quantification was used for comparison tests, and for optimizing the concentrations of TWEEN, ethanol and NaCl in the binding buffer.

### Results

Table 1. shows that varying levels of DNA yield are associated with varying compositions of binding buffer when TWEEN 20 is constant at 20%. In particular, Buffer ID Numbers 5 and 6 demonstrate high levels of DNA recovery consistent with efficient purification.

### Example 2

### Nucleic Acid Yield after Purification with 20%, 25%, and 30% TWEEN 20 (Polyoxyethylene Sorbitan Monolaurate)

### Sample Origin

Experiments were performed on aliquots of human white blood cell lysate prepared from whole blood, and stored as frozen stock samples. In Example 2 the identical lysate sample was used for all comparisons.

### Purification Protocol

In experimental Example 2, DNA yield using exemplary binding buffer compositions were compared using varying amounts of TWEEN 20, with 20% ethanol, and varying amounts of salt (Table 2). The magnetic bead (i.e., carboxyl coated paramagnetic particle) suspension solution was 40 μL microliter beads, 10 mM TRIS, and 3600 μL buffer (TWEEN buffer) for a 1:10 dilution of the beads in final buffer. The reaction mixture was 50 μL sample lysate, and 100 μL magnetic bead suspension. The mixture was incubated for 10 minutes, whereupon the beads were separated and washed 3 times with 500 μL 70% ethanol. The beads were then dried for 5 minutes before elution into 50 μL distilled water at 55° C. for 5 minutes. Nucleic acid purification has also been achieved, e.g., using a similar protocol involving silica based paramagnetic particles.

TABLE 2

| Buffer ID Number | 7 | 8 | 9 | 10 |
|---|---|---|---|---|
| Tween 20 | 20% | 20% | 30% | 25% |
| Ethanol | 20% | 20% | 20% | 20% |
| NaCl | 2.5M | 3.0M | 2.5M | 2.5M |
| DNA yield (ng. μL) | 41.7 | Precip | Precip | 39.6 |

### DNA Detection

Eluted DNA was then quantitated using a UV spectrophotometer. Absorbance at A260 wavelength was recorded. DNA quantification was used for comparison tests, and for optimizing the concentrations of TWEEN, ethanol and NaCl in the binding buffer.

### Results

Table 2 shows that varying levels of DNA yield are associated with varying compositions of binding buffer when TWEEN 20 varies between 20% and 30%, and ethanol is constant at 20%. In particular, Buffer ID Numbers 7 and 10 demonstrate high levels of DNA recovery consistent with efficient purification. By comparison, Buffer ID Numbers 8 and 9 yielded no measurable DNA upon purification because of NaCl precipitation.

### Example 3

### Comparison of TWEEN-Based Binding Buffer and Qiagen-Based Nucleic Acid Purification Methods for Influenza A Virus Detection

This example describes a comparison of two procedures, i.e., TWEEN-based binding buffer vs Qiagen-based methods, for the purification of nucleic acid for the detection of influenza A virus in human clinical samples.

### Sample Origin and Handling

The Naval Health Research Center (NHRC, San Diego, Calif.). NHRC supplied human respiratory specimens (throat swabs, nasal swabs, nasal wash specimens) collected and archived from various U.S. military bases from 1999 through 2005. Clinical swab samples were stored in Viral Transport Media (VTM).

### Purification Protocol

### a.) Qiagen-Based Purification

Clinical swab samples in Viral Transport Media (VTM) (1 mL) were passed over a 0.2 micron filter, which was then subjected to bead beating in a small amount of lysis buffer. The VTM/nasal matter was transferred to Qiagen kits. The resulting viral lysate was then prepared for analysis using the Qiagen QiaAmp Virus kit (Valencia, Calif.). Both manual (mini spin) kits and QIA Amp Virus BioRobot MDx kits were used per manufacturer's instructions. Robotic-based isolations were done on both the Qiagen MDx robot and Qiagen BioRobot 8000 platforms.

### b.) TWEEN-based Binding Buffer Purification

### Preparation of Magnetic Beads

One mL of Sera-mag carboxylated stock beads (5%) from Seradyn (Indianapolis, Ind.) was washed 3 times with 10 mM Tris buffer (pH8.0), and resuspended in 1 mL of 10 mM Tris buffer (pH 8.0).

### Preparation of the Magnetic Bead and Binding Buffer Mixture

The beads were then mixed with a binding buffer consisting of TWEEN 20, ethanol and salt in a mixture (Table 3.). Using Sera-mag beads, the resuspended 1 mL beads were mixed with 9 mL binding buffer consisting of 20% ethanol, 20% Tween 20, and 2.5M NaCl. The final concentration of the beads after washing the beads was 0.5 mg/mL.

TABLE 3
Binding Buffer

| Components | |
|---|---|
| Tween | 20% (variable) |
| (Binding Buffer) | |
| ETOH | 20% |
| (Binding Buffer) | |
| NaCl | 2.5 M (variable) |
| (Binding Buffer) | |
| PEG | 0 |
| (Binding Buffer) | |
| Bead concentration | 0.5 mg/mL |
| (Binding Buffer) | |
| Optional alternative | ETOH, NaCl |
| precipitating agents | |
| Tween | 0 |
| (Wash Buffer) | |
| Lysis Buffer | Lysis Buffer from Qiagen DNeasy |
| | kit or Ambion MagMAX lysis soln. |
| Wash Buffers | 70% ETOH, 2 times, no resuspension |

Lysis and Binding Nucleic Acid onto the Beads

Cells from the various sample sources were then lysed. Tested lysis buffers included lysis buffers from Qiagen DNeasy tissue kit (Valencia, Calif.), and Ambion MagMAX lysis solution (Austin, Tex.). Next, the lysate was mixed with the beads/binding buffer suspension at 1:1.5 volume ratios in an Eppendorf tube or a deep well plate (Table 4.). In the binding step, the TWEEN 20% was slightly less due to addition of 1 mL of beads in Tris buffer to each 9 mL of binding buffer to make bead/binding buffer suspension, which is then added at a 1.5:1 ratio to lysate. The mixture was then incubated at room temperature for 5 minutes.

TABLE 4
Reaction Mixture

| Components | |
|---|---|
| Tween | 12% (if none in |
| (binding step) | lysis buffer) |
| NaCl | 1.5 M (if none |
| (binding step) | in lysis buffer) |
| PEG | 0 |
| (binding step) | |
| Optional alternative | ETOH, NaCl |
| precipitating agents | |
| Tween | 0 |
| (Wash Buffer) | |
| Lysis Buffer | Lysis Buffer from Qiagen DNeasy |
| | kit or Ambion MagMAX lysis soln. |
| Wash Buffers | 70% ETOH, 2 times, no resuspension |
| Binding step time | 5′ |
| Bead concentration in | 0.5 mg/mL |
| Binding Buffer | |

### Washing the Beads

The reaction mixture of sample lysate and beads/binding buffer was then put into a magnetic separator where the beads move to the side of the tube by the magnet, allowing the remaining lysate (minus the nucleic acids) to be removed. The beads containing bound nucleic acids from the lysate were washed twice using 1 mL 70% ethanol. Resuspension of the beads during the wash was not necessary. The washed beads were then dried at room temperature for 5 minutes.

### Elution of Isolated Nucleic Acid

The washed beads were resuspended into 100 μL of elution buffer. The suspension of beads and elution buffer was incubated at 55° C. for 5 minutes, and the beads were separated from the solution using a magnetic separator. Finally, the solution was removed and stored at −20° C. until further use in downstream analyses.

### Detection Protocol

### DNA Detection

Eluted nucleic acid using the TWEEN-based binding buffer method and the Qiagen-based method was then quantitated using a UV spectrophotometer. Dilutions from the sample stocks were prepared for subsequent analysis by RT-PCR.

PCR Primer design

A surveillance panel of eight primer pairs was selected comprising one pan-influenza primer pair targeting the PB1 segment, five pan-influenza A primer pairs targeting NP, M1, PA and the NS segments, and two pan-influenza B primer pairs targeting NP and PB2 segments. All primers used had a thymine nucleotide at the 5′-end to minimize addition of non-templated adenosines during amplification using Taq polymerase. (Brownstein, M J et al., Modulation of non-templated nucleotide addition by Taq DNA polymerase: primer modifications that facilitate genotyping. Biotechniques 20, 1004-6, 1008-10 (1996)).

### Reverse Transcription PCR(RT-PCR)

One-step RT-PCR was performed in a reaction mix consisting of 4 U of AmpliTaq Gold (Applied Biosystems, Foster City, Calif.), 20 mM Tris (pH 8.3), 75 mM KCl, 1.5 mM MgCl₂, 0.4 M betaine, 800 μM mix of dATP dGTP dCTP and dTTP (Bioline USA Inc., Randolph, Mass.), 10 mM dithiothreitol, 100 ng sonicated polyA DNA (Sigma Corp., St Louis, Mo.), 40 ng random hexamers (Invitrogen Corp.), 1.2 U Superasin (Ambion Corp, Austin, Tex.), 400 ng T4 gene 32 protein (Roche Diagnostics Corp., Indianapolis, Ind.), 2 U Superscript III (Invitrogen Corp, Carlsbad Calif.), 20 mM sorbitol (Sigma Corp.) and 250 nM of each primer. 5 microliters of elutant from the Qiagen kits was used in a 50 microliter total reaction volume. The following RT-PCR cycling conditions were used: 60° C. for 5 min, 4° C. for 10 min, 55° C. for 45 min, 95° C. for 10 min, followed by 8 cycles of 95° C. for 30 seconds, 48° C. for 30 seconds, and 72° C. for 30 seconds, with the 48° C. annealing temperature increasing 0.9° C. each cycle. The PCR was then continued for 37 additional cycles of 95° C. for 15 seconds, 56° C. for 20 seconds, and 72° C. for 20 seconds. The RT-PCR cycle ended with a final extension of 2 minutes at 72° C. followed by a 4° C. hold.

### Mass Spectrometry and Base Composition Analysis

Following amplification, 15 μL aliquots of each PCR reaction were desalted and purified using a weak anion exchange protocol. Accurate mass (±1 ppm), high-resolution (M/dM>100,000 FWHM) mass spectra were acquired for each sample using high-throughput ESI-MS protocols described previously. (Hofstadler, S A et al., TIGER: the universal biosensor. Inter. J. Mass Spectrom. 242, 23-41 (2005)). For each sample, approximately 1.5 μL of analyte solution was consumed during the 74-second spectral acquisition. Raw mass spectra were post-calibrated with an internal mass standard and deconvolved to monoisotopic molecular masses. Unambiguous base compositions were derived from the exact mass measurements of the complementary single-stranded oligonucleotides. (Muddiman, D C et al., Length and Base Composition of PCR-Amplified Nucleic Acids Using Mass Measurements from Electrospray Ionization Mass Spectrometry. Anal. Chem. 69, 1543-1549 (1997). Quantitative results were obtained by comparing the peak heights with an internal PCR calibration standard present in every PCR well at 100 molecules. (Hofstadler, S A et al., TIGER: the universal biosensor. Inter. J. Mass Spectrom. 242, 23-41 (2005)).

### Results

Table 5. shows a comparison of results obtained for influenza A virus detection comparing TWEEN-based binding buffer and Qiagen-based methods of nucleic acid purification from human clinical samples. Column 1. indicates each sample's ID number. Columns 2 and 3 indicate the species and strain, respectively, of influenza A virus detected in the sample, if any. Column 4 indicates the relative amount of influenza A virus in each sample. Column 5 indicates whether sample preparation by TWEEN-based binding buffer methods and Qiagen-based methods are in accord. As can be seen from Table 5. column 5, all samples in this Example 3 showed full concordance in influenza A virus detection from human clinical samples comparing both methods of nucleic acid preparation.

TABLE 5

| | | | | detection |
|---|---|---|---|---|
| | | | | match |
| | | | | between |
| | | | | TWEEN |
| | | | | and |
| | | | Count | QIAGEN |
| Sample ID | Species | Strain | Est. | methods? |
| TGR1014 | Influenza A | A/NEW YORK/153/1999(H3N2) | >3000 | YES |
| | virus | | | |
| TGR1059 | Negative | null | 0 | YES |
| TGR1060 | Negative | null | 0 | YES |
| TGR1062 | Negative | null | 0 | YES |
| TGR1063 | Influenza A | A/NEW YORK/153/1999(H3N2) | 298.795 | YES |
| | virus | | | |
| TGR1066 | Negative | null | 0 | YES |
| TGR1067 | Negative | null | 0 | YES |
| TGR1068 | Negative | null | 0 | YES |
| TGR1069 | Influenza A | A/CANTERBURY/67/2005(H3N2) | >3000 | YES |
| | virus | | | |
| TGR1070 | Influenza A | A/NEW YORK/386/2004(H3N2) | >3000 | YES |
| | virus | | | |
| TGR1071 | Influenza A | A/CANTERBURY/101/2004(H3N2) | >3000 | YES |
| | virus | | | |
| TGR1072 | Influenza A | A/NEW YORK/135/2002(H3N2) | >3000 | YES |
| | virus | | | |
| TGR1075 | Influenza A | A/NEW YORK/380/2004(H3N2) | 385.128 | YES |
| | virus | | | |
| TGR1076 | Influenza A | A/NEW YORK/373/2005(H3N2) | >3000 | YES |
| | virus | | | |
| TGR1077 | Influenza A | A/NEW YORK/405/2002(H3N2) | >3000 | YES |
| | virus | | | |
| TGR1078 | Influenza A | A/CANTERBURY/418/2003(H3N2) | >3000 | YES |
| | virus | | | |
| TGR1079 | Influenza A | A/NEW YORK/153/1999(H3N2) | >3000 | YES |
| | virus | | | |
| TGR1080 | Negative | null | 0 | YES |
| TGR1081 | Negative | null | 0 | YES |
| TGR1082 | Negative | null | 0 | YES |
| TGR1085 | Influenza A | A/NEW YORK/76/2002(H3N2) | >3000 | YES |
| | virus | | | |
| TGR1086 | Influenza A | A/CANTERBURY/418/2003(H3N2) | >3000 | YES |
| | virus | | | |
| TGR1090 | Influenza A | A/NEW YORK/373/2005(H3N2) | >3000 | YES |
| | virus | | | |
| TGR1092 | Influenza B | ITCF-11605P2 | 1026.64 | YES |
| | virus | | | |
| TGR1097 | Influenza A | A/NEW YORK/461/2005(H3N2) | >3000 | YES |
| | virus | | | |
| TGR1104 | Influenza A | A/NEW YORK/386/2004(H3N2) | >3000 | YES |
| | virus | | | |
| TGR1107 | Influenza A | A/NEW YORK/372/2004(H3N2) | >3000 | YES |
| | virus | | | |
| TGR1110 | Negative | null | 0 | YES |
| TGR1111 | Influenza A | A/NEW YORK/330/1998(H3N2) | >3000 | YES |
| | virus | | | |
| TGR1113 | Influenza A | A/NEW YORK/182/2000(H3N2) | >3000 | YES |
| | virus | | | |
| TGR1114 | Influenza A | A/NEW YORK/440/2000(H3N2) | >3000 | YES |
| | virus | | | |
| TGR1115 | Influenza A | A/NEW YORK/440/2000(H3N2) | >3000 | YES |
| | virus | | | |
| TGR1116 | Negative | null | 0 | YES |
| TGR1130 | Influenza A | A/NEW YORK/153/1999(H3N2) | 252.3 | YES |
| | virus | | | |
| TGR1133 | Negative | null | 0 | YES |
| TGR1134 | Negative | null | 0 | YES |
| TGR1139 | Influenza A | A/NEW YORK/95/2002(H3N2) | >3000 | YES |
| | virus | | | |
| TGR1141 | Negative | null | 0 | YES |
| TGR1142 | Negative | null | 0 | YES |
| TGR1143 | Negative | null | 0 | YES |
| TGR1144 | Influenza A | A/NEW YORK/153/1999(H3N2) | >3000 | YES |
| | virus | | | |
| TGR1145 | Influenza A | A/CANTERBURY/418/2003(H3N2) | >3000 | YES |
| | virus | | | |
| TGR1146 | Influenza A | A/CANTERBURY/418/2003(H3N2) | >3000 | YES |
| | virus | | | |
| TGR1147 | Negative | null | 0 | YES |
| TGR1149 | Negative | null | 0 | YES |
| TGR1150 | Influenza A | A/NEW YORK/76/2002(H3N2) | >3000 | YES |
| | virus | | | |
| TGR1151 | Influenza A | A/NEW YORK/250/1998(H3N2) | >3000 | YES |
| | virus | | | |
| TGR1152 | Influenza A | A/NEW YORK/250/1998(H3N2) | >3000 | YES |
| | virus | | | |
| TGR1156 | Influenza A | A/CANTERBURY/101/2004(H3N2) | >3000 | YES |
| | virus | | | |
| TGR1158 | Influenza A | A/NEW YORK/373/2005(H3N2) | 377.586 | YES |
| | virus | | | |
| TGR1159 | Influenza B | ITCF-49120P2 | 1797.41 | YES |
| | virus | | | |
| TGR1161 | Negative | null | 0 | YES |
| TGR1173 | Influenza A | A/NEW YORK/386/2004(H3N2) | >3000 | YES |
| | virus | | | |
| TGR1174 | Influenza A | A/CANTERBURY/418/2003(H3N2) | 662.509 | YES |
| | virus | | | |
| TGR1175 | Influenza A | A/CANTERBURY/418/2003(H3N2) | >3000 | YES |
| | virus | | | |
| TGR1176 | Influenza A | A/CANTERBURY/418/2003(H3N2) | >3000 | YES |
| | virus | | | |
| TGR1177 | Negative | null | 0 | YES |
| TGR1178 | Negative | null | 0 | YES |
| TGR1179 | Negative | null | 0 | YES |
| TGR1181 | Influenza B | B/IBIS_REFERENCE_STANDARD/ | >3000 | YES |
| | virus | 2006 | | |
| TGR1182 | Influenza A | A/NEW YORK/440/2000(H3N2) | >3000 | YES |
| | virus | | | |
| TGR1183 | Influenza A | A/CANTERBURY/8/2000(H1N1) | 533.416 | YES |
| | virus | | | |
| TGR1184 | Influenza A | A/CHICKEN/YUNAN/3/01(H9N2) | >3000 | YES |
| | virus | | | |
| TGR1185 | Negative | null | 0 | YES |
| TGR1186 | Negative | null | 0 | YES |
| TGR1187 | Negative | null | 0 | YES |
| TGR1188 | Influenza A | A/NEW YORK/153/1999(H3N2) | 1083.35 | YES |
| | virus | | | |
| TGR1191 | Negative | null | 0 | YES |
| TGR1192 | Negative | null | 0 | YES |
| TGR1193 | Negative | null | 0 | YES |
| TGR1201 | Influenza A | A/CANTERBURY/418/2003(H3N2) | >3000 | YES |
| | virus | | | |
| TGR1202 | Influenza A | A/CANTERBURY/418/2003(H3N2) | >3000 | YES |
| | virus | | | |
| TGR1204 | Negative | null | 0 | YES |
| TGR1205 | Negative | null | 0 | YES |
| TGR1206 | Negative | null | 0 | YES |
| TGR1210 | Influenza A | A/NEW YORK/382/2005(H3N2) | 114.964 | YES |
| | virus | | | |
| TGR1214 | Influenza A | A/CANTERBURY/418/2003(H3N2) | 688.356 | YES |
| | virus | | | |
| TGR1215 | Influenza A | A/NEW YORK/95/2002(H3N2) | >3000 | YES |
| | virus | | | |
| TGR1216 | Negative | null | 0 | YES |
| TGR1217 | Negative | null | 0 | YES |
| TGR1218 | Negative | null | 0 | YES |
| TGR1244 | Influenza B | B/SICHUAN/379/99 | 321.52 | YES |
| | virus | | | |
| TGR1270 | Negative | null | 0 | YES |

### Example 4

### Nucleic Acid Purification From Bacillus thuringiensis

A sample of 3 mL of whole blood containing 500 colony forming units (CFU) of Bacillus thuringiensis was processed using the magnetic bead protocol as follows:
1. 15 mL conical tubes were prepared for bead beating by adding:
   a. 1 mL 0.1 mm zirconium/silica beads
   b. 1 mL 0.5 mm zirconium/silica beads
   c. 300 μL protease
2. 50 ml conical tubes were prepared for magnetic bead binding:
   a. 1 mL of carboxylated magnetic beads (Seradyn, Inc.) at 2 mg/mL
   b. 13 mL binding buffer (20% ethanol, 20% Tween 20, and 2.5M NaCl)
3. 3 mL sample was added to each 15 mL conical tube containing beads and protease.
4. 3.6 mL lysis buffer was added to each 15 mL conical tube.
5. Bead beating was carried out using an MP FastPrep instrument (MP Biomedicals United States, Solon, Ohio)
   a. Time: 3×60 seconds.
   b. Speed: 6.5 M/seconds.
6. The tubes were transferred to a 56° C. water bath
   a. Incubated for 30 minutes.
7. The tubes were centrifuged for 1 minute at 3000 rpm
8. The supernatant was transferred to a 50 mL conical tube containing carboxylated magnetic beads in binding buffer (comprising 20% ethanol, 20% Tween 20, and 2.5M NaCl), taking care to leave the bead beating beads behind.
9. The tubes were gently inverted for 15 minutes to allow binding of nucleic acid to the beads.
10. The 50 mL conicals were centrifuged for 3 minutes at 5000 rpm
11. The supernatant was poured off leaving the magnetic bead pellet behind.
   a. Any remaining supernatant was removed with a pipette leaving only the magnetic beads
12. 1 mL of binding buffer (comprising 20% ethanol, 20% Tween 20, and 2.5M NaCl was added to the magnetic bead pellet.
13. The magnetic bead pellet was resuspended with a pipette and transferred to a deep well 96-well plate.
14. The beads containing bound nucleic acid were washed in 1 mL wash buffer 1 (Qiagen buffer AW1), 1 mL wash buffer 2 (Qiagen AW2), and eluted in 250 microliters of elution buffer (Qiagen buffer AE) using the KingFisher 96 instrument (Thermo Scientific)

A sample of 3 mL of whole blood containing 500 colony forming units (CFU) of Bacillus thuringiensis was also processed using a Qiagen QIAamp DNA Blood Midi column procedure following the manufacturer's instructions for whole blood.

Results show that the Ibis magnetic bead isolation of Bacillus thuringiensis DNA resulted in detection at 2 cfu/ml, while the Qiagen isolation only detected at the 31 cfu/ml level using the Ibis T5000 biosensor (Table 6. T5000 Results: Bacillus thuringiensis in whole blood.). In addition, the direct measurement of total DNA present (both human DNA from blood and DNA from Bacillus thuringiensis) was significantly greater for the Ibis magnetic bead method when compared to the Qiagen Midi procedure (Table. 7. Total DNA present (by direct UV measurement)).

TABLE 6

| | Qiagen | Ibis |
|---|---|---|
| cfu/ml | genomes | genomes |
| Spike | detected | detected |
| 500 | 468 | 3557 |
| 250 | 241 | 2296 |
| 125 | 97 | 1811 |
| 62.5 | 42 | 1368 |
| 31 | 28 | 694 |
| 16 | ND | 259 |
| 8 | ND | 146 |
| 4 | ND | 54 |
| 2 | ND | 30 |
| 1 | ND | ND |
| 0.5 | ND | ND |
| Blood Control | NA | NA |

TABLE 7
Total DNA Yield

| CFU/ml | Method B | Method A |
|---|---|---|
| 500 | 2.85 ug | 29.6 ug |
| 250 | 2.43 ug | 26.0 ug |
| 125 | 2.24 ug | 46.8 ug |
| 62 | 3.26 ug | 47.4 ug |
| 31 | 3.82 ug | 54.2 ug |
| 16 | 2.80 ug | 22.0 ug |
| 8 | 1.77 ug | 57.0 ug |
| 4 | 2.94 ug | 72.0 ug |
| 2 | 2.56 ug | 30.6 ug |
| 1 | 3.29 ug | 30.2 ug |
| 0.5 | 2.92 ug | 48.2 ug |
| Blood Control | 3.62 ug | 48.0 ug |

### Example 5

### Comparison of Ibis' Magnetic Bead Nucleic Acid Isolation Process to Qiagen QiaAmp MinElute Virus Spin Kit for Isolation of Influenza A Virus: Further Illustration of RNA Isolation

A 1:2 dilution series of samples of Influenza A Virus (an RNA virus) was prepared. 200 microliter samples were used for viral genome isolation. For both methods, viral lysis was carried out as described in the Qiagen QIAamp MinElute Virus Spin kit. Following lysis, the RNA genome was isolated using either an Ibis' magnetic bead-based isolation process as described herein or with Qiagen's QIAamp MinElute Virus Spin kit according to the manufacturer's instructions. Following isolation, samples were analyzed using a Flu 8 PP kit (Ibis Biosciences) and the T5000 system.

The results show that Influenza A RNA isolated with the Ibis isolation process gave signal at the 1024× dilution, while the QIAamp Virus Spin kit gave a T5000 signal at the 128× dilution, an 8× difference (Table 8. T5000 Results: Influenza A Virus).

TABLE 8
Genomes/Well Detected

| Sample | Dilution | QIAamp Virus Spin kit | Ibis Magnetic Beads |
|---|---|---|---|
| 1 | No dilution | 7026 | 23147 |
| 2 | 2x | 4535 | 12669 |
| 3 | 4x | 1652 | 5125 |
| 4 | 8x | 778 | 1638 |
| 5 | 16x | 378 | 1057 |
| 6 | 32x | 171 | 299 |
| 7 | 64x | 83 | 166 |
| 8 | 128x | 23 | 88 |
| 9 | 256x | ND | 34 |
| 10 | 512x | ND | 22 |
| 11 | 1024x | ND | 18 |
| 12 | Water control | ND | ND |

### REFERENCES

1. DeAngelis, M. M., Wang, D. G., and Hawkins, T. L. (1995) Nucleic Acids Res 23, 4742-3.
2. Elkin, C. J., Richardson, P. M., Fourcade, H. M., Hammon, N. M., Pollard, M. J., Predki, P. F., Glavina, T., and Hawkins, T. L. (2001) Genome Res 11, 1269-74.
3. Hawkins, T. L., O′Connor-Morin, T., Roy, A., and Santillan, C. (1994) Nucleic Acids Res 22, 4543-4.
4. U.S. Pat. No. 5,705,628 (Hawkins)
5. U.S. Pat. No. 5,898,071 (Hawkins)
6. US Published App. US 2006/0078923 A1 (McKernan)
7. US Published App. US 2006/0147957 A1 (Qian)
8. US Published App. US 2006/0177836 (McKernan)
9. US Published App. US 2006/0024701 A1 (McKernan)
10. US Published App. US 2006/0240448 A1

Having fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

1. A method for nucleic acid purification, comprising:
a) combining a binding buffer comprising polyoxyethylene sorbitan monolaurate, at least one alcohol and at least one salt with at least one paramagnetic particle to generate a suspension;
b) combining at least one sample comprising at least one nucleic acid with said suspension, wherein said paramagnetic particle reversibly captures said nucleic acid to generate a combination comprising said paramagnetic particle with said captured nucleic acid; and,
c) separating said paramagnetic particle with said captured nucleic acid from one or more other components of the combination using a magnetic separator, thereby purifying said nucleic acid.

2. The method of claim 1, comprising washing said paramagnetic particle with said captured nucleic acid with a wash buffer.

3. The method of claim 1, wherein said nucleic acid non-covalently binds to said paramagnetic particle.

4. The method of claim 1, wherein said paramagnetic particle comprises a carboxyl coated paramagnetic particle or a silica based paramagnetic particle.

5. The method of claim 1, comprising combining said sample with a lysis buffer to generate a lysate.

6. The method of claim 5, wherein b) comprises combining said lysate with said suspension.

7. The method of claim 1, comprising releasing said captured nucleic acid from said paramagnetic particle to generate released nucleic acid.

8. The method of claim 7, wherein said releasing comprises incubating said paramagnetic particle with said captured nucleic acid with an elution buffer.

9. The method of claim 7, comprising separating said released nucleic acid from said paramagnetic particle using said magnetic separator.

10. A method for nucleic acid purification, comprising:
a) obtaining a sample comprising or suspected of comprising at least one nucleic acid;
b) providing:
i) a solution comprising at least one paramagnetic particle;
ii) a solution comprising a binding buffer comprising polyoxyethylene sorbitan monolaurate, at least one alcohol and at least one salt;
iii) a lysis buffer;
iv) a magnetic separator;
v) a wash buffer; and
vi) an elution buffer; and
c) combining said binding buffer with said at least one paramagnetic particle to generate a suspension;
d) combining said sample with said lysis buffer to generate a lysate;
e) combining said suspension with said lysate to generate a combination;
f) placing said combination of said suspension with said lysate into a magnetic separator;
g) separating said combination of said suspension with said lysate from said at least one paramagnetic particle;
h) washing said at least one paramagnetic particle with said wash buffer;
i) incubating said at least one paramagnetic particle with said elution buffer; and
j) separating said at least one paramagnetic particle from said elution buffer using said magnetic separator.

11. The method of claim 10, wherein said solution comprising a binding buffer comprises at least 10% polyoxyethylene sorbitan monolaurate.

12. The method of claim 10, wherein said solution comprising a binding buffer comprises at least 20% polyoxyethylene sorbitan monolaurate by volume.

13. The method of claim 10, wherein said solution comprising a binding buffer comprising at least one alcohol comprises ethanol.

14. The method of claim 10, wherein said solution comprising a binding buffer comprises at least 10% ethanol by volume.

15. The method of claim 10, wherein said solution comprising a binding buffer comprises at least 20% ethanol by volume.

16. The method of claim 10, wherein said solution comprising a binding buffer comprising at least one salt comprises NaCl.

17. The method of claim 10, wherein said solution comprising a binding buffer comprising at least one salt comprises at least 1.0 M NaCl.

18. The method of claim 10, wherein said solution comprising a binding buffer comprising at least one salt comprises at least 2.0 M NaCl.

19. The method of claim 18, further comprising at least 10% polyoxyethylene sorbitan monolaurate by volume.

20. The method of claim 19, further comprising at least 10% ethanol by volume.

21. The method of claim 10, wherein said combination of said suspension with said lysate comprises at least 7.5% polyoxyethylene sorbitan monolaurate.

22. The method of claim 10, wherein said combination of said suspension with said lysate comprises at least 10% polyoxyethylene sorbitan monolaurate.

23. The method of claim 22, further comprising at least 1.5 M NaCl.

24. The method of claim 10, wherein said at least one nucleic acid is DNA.

25. The method of claim 10, wherein said at least one nucleic acid is RNA.

26. The method of claim 10, wherein said at least one nucleic acid is nucleic acid from a prokaryote.

27. The method of claim 10, wherein said at least one nucleic acid is nucleic acid from a eukaryote.

28. The method of claim 10, wherein said sample is from a biologic source.

29. The method of claim 10, wherein said sample is from a non-biological source.

30. The method of claim 10, wherein said combination is a reaction mixture generated by sequentially conducting steps a) to e).

31. The method of claim 10, wherein said paramagnetic particle comprises a carboxyl coated paramagnetic particle or a silica based paramagnetic particle.

32. A kit, comprising
a) a binding buffer, comprising:
i) polyoxyethylene sorbitan monolaurate;
ii) at least one alcohol; and
b) at least one paramagnetic particle.

33. The kit of claim 32, comprising one or more of:
c) a lysis buffer;
d) a reaction vessel;
e) a magnetic separator;
f) a wash buffer; or
g) an elution buffer.

34. The kit of claim 32, wherein said binding buffer comprises at least 10% polyoxyethylene sorbitan monolaurate by volume.

35. The kit of claim 32, wherein said binding buffer comprises at least 20% polyoxyethylene sorbitan monolaurate by volume.

36. The kit of claim 32, wherein said at least one alcohol comprises ethanol.

37. The kit of claim 36, wherein said at least one alcohol comprises at least 10% ethanol by volume.

38. The kit of claim 36, wherein said at least one alcohol comprises at least 20% ethanol by volume.

39. The kit of claim 32, wherein said binding buffer further comprises at least one salt.

40. The kit of claim 39, wherein said at least one salt is NaCl.

41. The kit of claim 39, wherein said at least one salt comprises at least 1.0 M NaCl.

42. The kit of claim 39, wherein said at least one salt comprises at least 2.0 M NaCl.

43. The kit of claim 42, wherein said binding buffer further comprises at least 10% polyoxyethylene sorbitan monolaurate by volume.

44. The kit of claim 43, wherein said binding buffer further comprises at least 10% ethanol by volume.

45. The kit of claim 32, further comprising instructions for using said kit on a computer readable medium.

46. The kit of claim 33, wherein said binding buffer, said at least one paramagnetic particle, said lysis buffer, said wash buffer and said elution buffer are provided in individual containers.

47. The kit of claim 33, wherein said wash buffer comprises at least 70% ethanol by volume.

48. The kit of claim 32, wherein said paramagnetic particle comprises a carboxyl coated paramagnetic particle or a silica based paramagnetic particle.

49. A composition comprising at least one paramagnetic particle in a binding buffer comprising 20% polyoxyethylene sorbitan monolaurate by volume, 20% ethanol by volume, and 2.5 M NaCl.
